(19) United States
(12) Patent Application Publication
(10) Pub. No.: US 2019/0216368 A1
(43) Pub. Date: Jul. 18, 2019

(54) METHOD OF PREDICTING DAILY ACTIVITIES PERFORMANCE OF A PERSON WITH DISABILITIES

(71) Applicants: Chih-Kuang Chen, Taipei City (TW); Chun-Hsien Chen, Taoyuan City (TW); Hsin-Yao Wang, Chiayi City (TW); Wan-Ying Lin, Taipei City (TW)

(72) Inventors: Chih-Kuang Chen, Taipei City (TW); Chun-Hsien Chen, Taoyuan City (TW); Hsin-Yao Wang, Chiayi City (TW); Wan-Ying Lin, Taipei City (TW)

(73) Assignees: CHANG GUNG MEMORIAL HOSPITAL, LINKOU, Taoyuan City (TW); Chang Gung University, Taoyuan City (TW)

(21) Appl. No.: 15/870,930

(22) Filed: Jan. 13, 2018

(51) Int. Cl. A61B 5/11; A61B 5/00

(52) U.S. Cl. CPC A61B 5/1118; A61B 5/1113; A61B 2503/08; A61B 5/1124; A61B 5/1123; A61B 2505/07; A61B 5/0022

(57) ABSTRACT

A method of predicting daily living activities performance of a person with disabilities includes establishing a rehabilitation assessments panel based on a plurality of rehabilitation evaluation scales and laboratory data; evaluating a plurality of persons with disabilities by the rehabilitation assessments panel; entering assessment results and the corresponding activities of daily living (ADL) performance into a machine learning platform; utilizing variable selection methods to select a plurality of variables having optimal classification performance from the rehabilitation assessments panel; executing a machine learning algorithm to create an ADL prediction model based on the selected variables; evaluating a participant in terms of the rehabilitation assessments panel; and entering assessment results into the ADL prediction model for calculation, thereby obtaining a prediction result of future ADL performance for the participant.

### BACKGROUND OF THE INVENTION

### 1. Field of the Invention

The invention relates to technologies of predicting post-stroke activities of daily living (ADL) of a person and more particularly to a method of correctly predicting post-stroke daily living activities of a person by establishing an ADL prediction model so that healthcare resources can be correctly allocated for optimized care of a post-stroke patient according to the prediction result of the ADL prediction model for the patient.

### 2. Description of Related Art

A person with disabilities is defined as a person loses some or all physical or mental functions so that his or her daily activities need to be taken care of by another person. Activities of daily living (ADL) refers to people's daily self care activities. The disability degree of a person can be evaluated by the ADL performance ability of a person, and it can be classified as mild, moderate and severe. It is estimated that there were about 670,000 persons with disabilities in Taiwan and about 410,000 persons of them were at least 65 years of age in year 2011. And, it is estimated that there will be about 860,000 persons with disabilities in Taiwan and about 600,000 persons of them are at least 65 years of age in year 2020.

Post-stroke persons having mild disability may quickly deteriorate into moderate or even severe disability if sufficient care is not provided to them. Life of a person having mild disability can be prolonged greatly due to the advancement of modern medicine technologies. There will be more elderly persons having moderate or severe disabilities in the future. And in turn, this will impose a greater burden on the society.

For overcoming the healthcare problem of persons with disabilities, the conventional method is to evaluate the daily activities of a person with disabilities by manually interpreting a particular rehabilitation assessment. However, the conventional method is disadvantageous owing to lacking a systematic evaluation method, poor clinical effectiveness, low correctness, inefficiency and unreliable reproducibility of interpretation results. Besides, it can not take advantage of the comprehensive data distribution patterns of multiple rehabilitation assessments as well as multiple laboratory data items, and it can not predict the future daily activities of a person with disabilities.

Thus, the need for improvement still exists.

### SUMMARY OF THE INVENTION

Therefore one object of the invention is to provide a method of predicting daily living activities performance of a person with disabilities by using a rehabilitation assessments panel based on a plurality of rehabilitation evaluation scales and laboratory data; evaluating a plurality of persons with disabilities with the rehabilitation assessments panel; entering assessment results and their corresponding ADL performance into a machine learning platform; utilizing variable selection methods to select a plurality of variables having optimal classification performance from the rehabilitation assessments panel; executing a machine learning algorithm to create an ADL prediction model based on the selected variables; evaluating a participant in terms of the rehabilitation assessments panel; and entering assessment results into the ADL prediction model for calculating, thereby obtaining a prediction result of future ADL performance for the participant.

The invention has the following advantages and benefits in comparison with the conventional art: A correct prediction of ADL of a person with disabilities can be made. Healthcare resources can be correctly allocated for optimized care of the person according to the prediction result. The ADL prediction model takes advantage of comprehensive data distribution patterns of multiple rehabilitation assessments, which can provide rich rehabilitation information to medical employees for understanding the ADL and health status of persons with disabilities. The more rehabilitation assessments a person takes, the more completeness of his/her rehabilitation evaluation will be. In comparison with manual interpretation of a plurality of rehabilitation assessments of a person with disabilities, the efficiency and the accuracy of ADL prediction model are significantly increased. Moreover, the ADL prediction model can be easily copied to other computers for massive applications.

The above and other objects, features and advantages of the invention will become apparent from the following detailed description taken with the accompanying drawings.

### BRIEF DESCRIPTION OF THE DRAWINGS

FIG. 1 is a flow chart of a method of predicting daily activities performance of a person with disabilities according to the invention;

FIG. 2 plots true positive rate versus pseudo positive rate for receiver operating characteristic (ROC) curve utilized in the IADL prediction model of the invention;

FIG. 3 is a table of area under ROC curve in terms of each machine learning algorithms including, Logistic Regression (LR), Decision Tree (DT), Random Forest (RF) and K Nearest Neighbor (KNN), according to the invention; and

FIG. 4 is a table showing the prediction performance of the rehabilitation assessments panel combined with a machine learning algorithm and the prediction performance of the single scales in term of area under the curve (AUC) average and AUC standard deviation according to the invention.

### DETAILED DESCRIPTION OF THE INVENTION

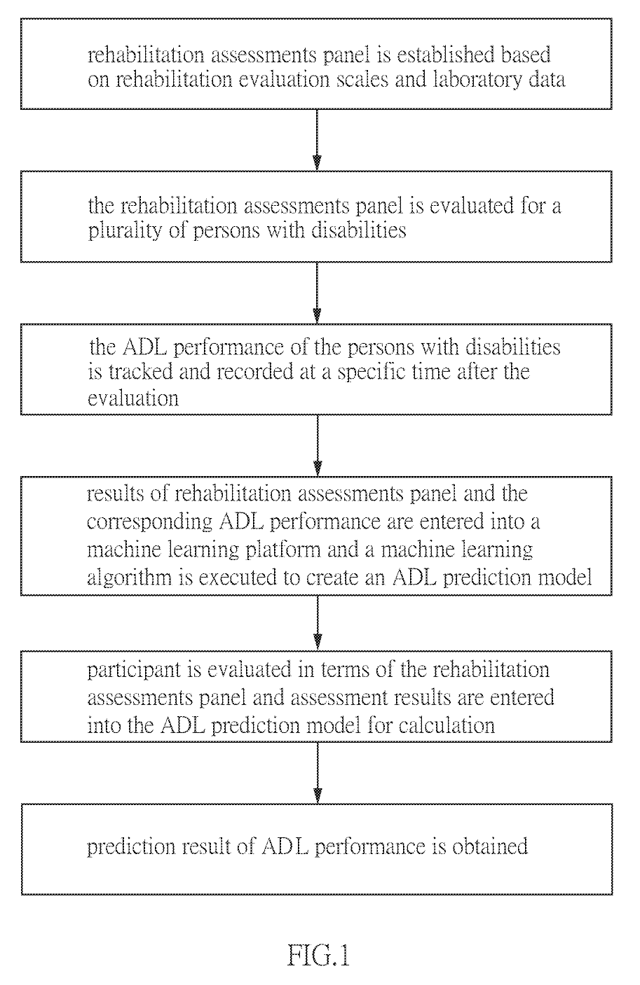

Referring to FIG. 1, a flow chart of a method of predicting daily activities of a person with disabilities in accordance with the invention comprises the following steps as described in detail below.

A rehabilitation assessments panel is established based on a plurality of rehabilitation evaluation scales and laboratory data.

The rehabilitation assessments panel is evaluated for a plurality of persons with disabilities.

The ADL performance of the persons with disabilities is tracked and recorded at a specific time after the evaluation.

Evaluation results and the corresponding ADL performance are entered into a machine learning platform.

A variable selection method is utilized to select a plurality of variables having optimal classification performance among the rehabilitation assessments panel. A machine learning algorithm is executed to create an ADL prediction model based on the selected variables.

A subject participating the test is evaluated in terms of the rehabilitation assessments panel. The assessment results are entered into the ADL prediction model for calculation. And in turn, a prediction result of future ADL performance is obtained for the subject.

Preferably, the person participating in the test is notified of the prediction result so that subsequent actions may be taken for him/her.

Preferably, the length of time between the date of determining ADL and the date of evaluating rehabilitation evaluation scales is from two weeks to one year.

Preferably, the rehabilitation evaluation scales include Modified Rankin Scale (MRS), Barthel Index, Functional Oral Intake Scale (FOIS), Mini Nutrition Assessment (MNA), Euro QoL-5D, IADL Scale, Berg Balance Scale (BBS), Gait speed, Six Minutes Walking Test (6MWT), Fugl-Meyer Assessment (FMA), Mini-Mental State Examination (MMSE), Motor Activity Log (MAL), Concise Chinese Aphasia Test (CCAT), and any combinations thereof.

Preferably, the ADL performance is evaluated by using Barthel Index, IADL Scale or Modified Rankin Scale (MRS).

Preferably, the laboratory data include CBC, White Blood Cells Differential Counts, Total Protein, Albumin, Leukocyte Esterase, High-Sensitivity C-Reactive Protein (hsCRP), Procalcitonin, Erythrocyte Sedimentation Rate, Lactate, Lactate Dehydrogenase, Sugar, Nat, Ca²⁺, Cl⁻, Mg²⁺, Fe²⁺, Fe³⁺, Urea Nitrogen, Creatinine, Cystatin C, Bilirubin, Low Density Lipoprotein (LDL), High Density Lipoprotein (HDL), Triglyceride, Total cholesterol, blood sugar, Microalbumin, HbA1C, Homocysteine, Lipoprotein A, Uric acid, and any combinations thereof.

Preferably, the machine learning algorithms include Logistic Regression (LR), K Nearest Neighbor (KNN), Support Vector Machines (SVM), Artificial Neuron Network (ANN), Decision Tree (DT), Random Forest (RF), Bayesian Network, and any combinations thereof.

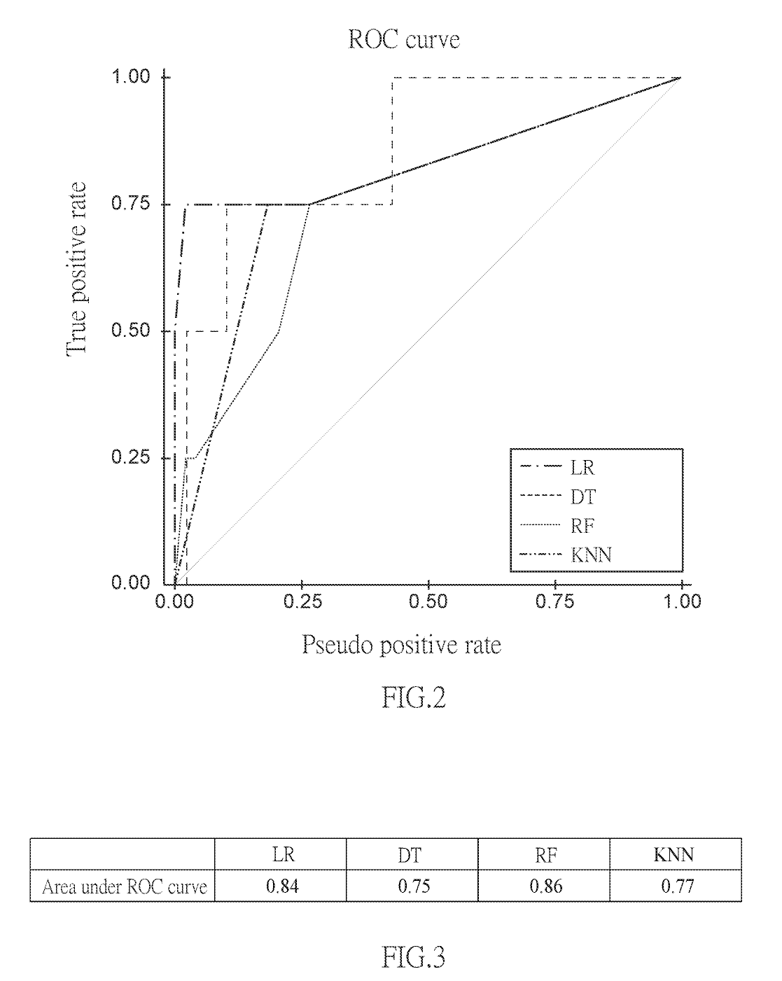

Referring to FIGS. 2 and 3 in conjunction with FIG. 1, the method of the invention is implemented below.

A rehabilitation assessments panel evaluated on patients suffering from stroke and seeking treatment at Chang Gung Memorial Hospital is taken as an embodiment of the invention. The rehabilitation assessment results of the patients associated with their corresponding ADL performance (i.e. IADL) are entered into the machine learning platform. Further, the machine learning algorithm is executed to create an ADL prediction model.

Conditions (including admission and exclusive) of an individual for the test and the number of samples:

Any patient suffering from stroke and seeking treatment at Chang Gung Memorial Hospital is appropriate as a candidate. Medical records of patients are checked to find 313 potential candidates. There is no need of recruiting candidates from outside patients.

Design and Method:

Clinical information contains the rehabilitation evaluation scales including Modified Rankin Scale (MRS), Barthel Index, Functional oral intake scale (FOIS), Mini Nutrition Assessment (MNA), Euro QoL-5D, IADL Scale, Berg Balance Scale (BBS), Gait speed, Six Minutes Walking Test (6MWT), Fugl-Meyer Assessment (FMA), Mini-Mental State Examination (MMSE), Motor Activity Log (MAL) and Concise Chinese Aphasia Test (CCAT).

The ADL performance of each candidate has been recorded at hospital admission and discharge respectively. The scores of the rehabilitation assessments panel are kept as evaluation data. The evaluation data is processed and its variables are selected from the rehabilitation assessments panel by executing a variable selection method based on the ADL such that optimal classification performance is achieved. Regarding the variable selection, a univariate analysis is conducted in the embodiment after preliminary data cleaning. An appropriate univariate statistical method (e.g., Chi-square test or t test) is selected based on the characteristic of the variable. As a result, MRS, BBS and IADL scale are selected. After the variable selection, they are entered into the machine learning platform which in turn executes a machine learning algorithm such as Logistic Regression (LR), Decision Tree (DT), Random Forest (RF), K Nearest Neighbor (KNN), Support Vector Machines (SVM) or Artificial Neuron Network (ANN) to create a prediction model.

Retrospective period of the embodiment: from March, 2014 to October, 2016.

Evaluation Results and Verification Method:

In the embodiment, data distributions of the assessments in the rehabilitation assessments panel are calculated. Further, prediction models are trained based on the variables and their associated data values. In the embodiment, the prediction capability of each ADL prediction model is evaluated. The prediction performance of each ADL prediction model is evaluated based on the ROC curve and the area under the ROC curve (AUC) is calculated accordingly.

Performance:

FIG. 2 plots true positive rate versus pseudo positive rate for receiver operating characteristic (ROC) curve utilized in IADL prediction model of the invention, and FIG. 3 is a table of the areas under ROC curve achieved by various machine learning algorithms including Logistic Regression (LR), Decision Tree (DT), Random Forest (RF) and K Nearest Neighbor (KNN) according to the invention. The performance of IADL prediction models can be evaluated in terms of AUC. As shown in FIG. 3, AUCs are 0.84 (LR), 0.75 (DT), 0.86 (RF) and 0.77 (KNN) respectively. It is concluded that all of the above machine learning algorithms are good, and LR as well as RF are preferred among them.

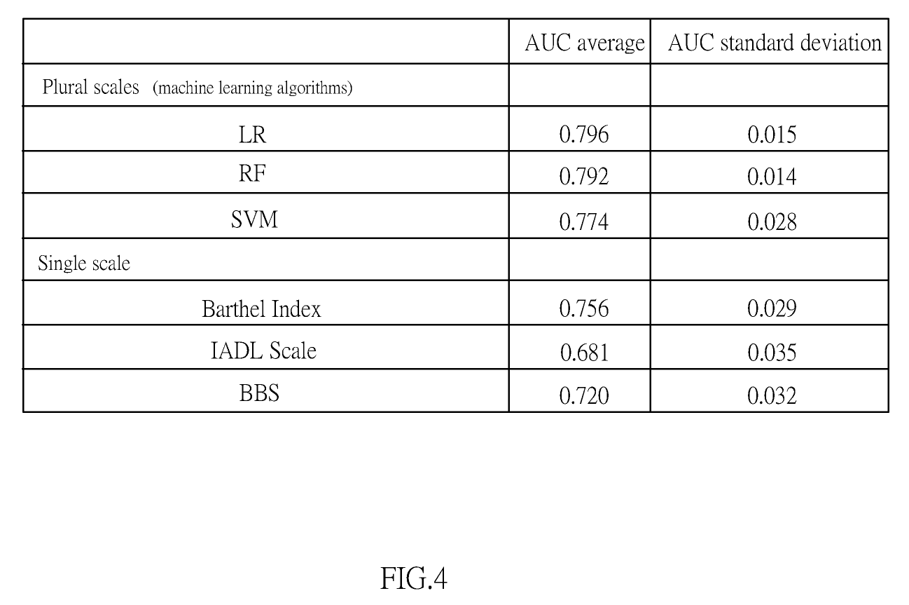

FIG. 4 is a table comparing the performances of various ADL prediction models based on the rehabilitation assessments panel combined with a machine learning algorithm versus single scales. The comparison is evaluated in terms of AUC average and AUC standard deviation according to the invention. Assessment values of a candidate at hospital admission are taken as a basis to predict ADL performance of the candidate at discharge. As shown in the figure, for LR, AUC average is 0.796 and AUC standard deviation is 0.015; for RF, AUC average is 0.792 and AUC standard deviation is 0.014; and for SVM, AUC average is 0.774 and AUC standard deviation is 0.028. The single scales include Barthel Index having an AUC average of 0.756 and an AUC standard deviation of 0.029; IADL Scale having an AUC average of 0.681 and an AUC standard deviation of 0.035; and BBS having an AUC average of 0.720 and an AUC standard deviation of 0.032.

In view of above description, it is found that AUC standard deviation for any of the prediction models based on the rehabilitation assessments panel (combined with any of the machine learning algorithms) is much less than that for one based on any of the single scales. It is also found that AUC average for any of the prediction models based on the rehabilitation assessments panel (combined with any of the machine learning algorithms) is greater than that for one based on any of single scales. This confirms that the performance of ADL prediction model can be increased greatly by adding a plurality of assessment scales to the rehabilitation assessments panel. Also, the rehabilitation assessments panel integrated with machine learning algorithms can greatly increase the performance of ADL prediction model.

The invention has the following characteristics and advantages: ADL can be predicted accurately by prediction models analyzing the rehabilitation assessments panel integrated with machine learning algorithms.

While the invention has been described in terms of preferred embodiments, those skilled in the art will recognize that the invention can be practiced with modifications within the spirit and scope of the appended claims.

What is claimed is:

1. A method of predicting daily activities performance of a person with disabilities comprising the steps of:
(1) establishing a rehabilitation assessments panel based on a plurality of rehabilitation evaluation scales and laboratory data;
(2) evaluating a plurality of persons with disabilities with the rehabilitation assessments panel;
(3) entering assessment results and the corresponding activities of daily living (ADL) performance into a machine learning platform;
(4) utilizing variable selection methods to select a plurality of variables having optimal classification performance from the rehabilitation assessments panel;
(5) executing a machine learning algorithm to create an ADL prediction model based on the selected variables;
(6) measuring a participant in terms of the rehabilitation measures panel; and
(7) entering assessment results into the ADL prediction model for calculation, thereby obtaining a prediction result of ADL performance.

2. The method of claim 1, wherein the ADL performance of persons with disabilities is tracked and recorded at a specific time after the evaluation at step (2).

3. The method of claim 1, wherein after obtaining a prediction result of ADL performance, a person participating in the test is notified of the prediction result so as to take subsequent actions.

4. The method of claim 1, wherein the length of time between the date of determining ADL performance and the date of evaluating rehabilitation evaluation scales is from two weeks to one year.

5. The method of claim 1, wherein the rehabilitation evaluation scales include Modified Rankin Scale (MRS), Barthel Index, Functional Oral Intake Scale (FOIS), Mini Nutrition Assessment (MNA), Euro QoL-5D, Instrumental Activities of Daily Living (IADL) Scale, Berg Balance Scale (BBS), Gait Speed, Six Minutes Walking Test (6MWT), Fugl-Meyer Assessment (FMA), Mini-Mental State Examination (MMSE), Motor Activity Log (MAL), Concise Chinese Aphasia Test (CCAT), and any combinations thereof.

6. The method of claim 1, wherein the ADL performance is evaluated by using Barthel Index, IADL Scale or Modified Rankin Scale (MRS).

7. The method of claim 1, wherein the laboratory data include CBC, White Blood Cells Differential Counts, Total Protein, Albumin, Leukocyte Esterase, High-Sensitivity C-Reactive Protein (hsCRP), Procalcitonin, Erythrocyte Sedimentation Rate, Lactate, Lactate Dehydrogenase, Sugar, Nat, K⁺, Ca²⁺, Cl⁻, Mg²⁺, Fe²⁺, Fe³⁺, Urea Nitrogen, Creatinine, Cystatin C, Bilirubin, Low Density Lipoprotein (LDL), High Density Lipoprotein (HDL), Triglyceride, Total cholesterol, blood sugar, Microalbumin, HbA1C, Homocysteine, Lipoprotein A, Uric acid, and any combinations thereof.

8. The method of claim 1, wherein the machine learning algorithms include Logistic Regression (LR), K Nearest Neighbor (KNN), Support Vector Machines (SVM), Artificial Neuron Network (ANN), Decision Tree (DT), Random Forest (RF), Bayesian Network, and any combinations thereof.
